(19) United States
(12) Patent Application Publication
(10) Pub. No.: US 2018/0251483 A1
(43) Pub. Date: Sep. 6, 2018

(54) THIOCYANATE SALTS FOR ANTI-INFLAMMATION

(71) Applicants: National Jewish Health, Denver, CO (US); Aeolus Pharmaceuticals, Inc., Mission Viejo, CA (US)

(72) Inventors: Brian J. Day, Denver, CO (US); John McManus, Mission Viejo, CA (US)

(21) Appl. No.: 15/782,359

(22) Filed: Oct. 12, 2017

Related U.S. Application Data
(63) Continuation of application No. PCT/US16/27348, filed on Apr. 13, 2016.
(60) Provisional application No. 62/146,901, filed on Apr. 13, 2015.

(51) Int. Cl. C07F 13/00; A61P 39/06; C01C 3/00

(52) U.S. Cl. CPC C07F 13/005; C01C 3/005; A61P 39/06

(57) ABSTRACT

Described herein, inter alia, are thiocyanate salt compositions and methods for treating or preventing inflammation using the same.

### CROSS-REFERENCES TO RELATED APPLICATIONS

This application claims the benefit of U.S. Provisional Application No. 62/146,901, filed Apr. 13, 2015, which is hereby incorporated by reference in its entirety and for all purposes.

### BACKGROUND OF THE INVENTION

Many of today's chronic inflammatory diseases were once considered diseases attributed to aging. However their continued growth has overwhelmed our health care system and is fueling our growing interest in developing better treatment options.

There are many different inflammatory diseases, yet all of them share the same underlying driver: an inappropriate inflammatory response or a body's immune response is out of balance. Typically, chronic and acute inflammatory diseases or disorders can be treated with antihistamines, anti-inflammatory drugs, corticosteroids or chondroprotective agents, depending on the nature and severity of the inflammation being treated. However, these treatments are not always efficacious in treating chronic and/or acute inflammation and can sometimes exhibit undesirable side-effects when used short or long term.

While treatment options have improved over the last decade for many inflammatory conditions, not all inflammatory conditions have made the same advances in the discovery and development of new effective therapies.

For example, more than 25 million Americans suffer from asthma, which is twice as many as in 1990. The severity of the disease is on the rise with more people dying from asthma in 2000 than in 1970. The development in finding novel treatment options for lung inflammation as a result of asthma still remains a big challenge. Although many drugs are available as inhalers to provide direct drug delivery to the lungs not all of these drugs are effective to relieve symptoms without exhibiting undesirable side-effects.

Another example is the development of treatment options for cystic fibrosis (CF). CF is a life-threatening, genetic disease that primarily affects the lungs and digestive system. It is found in about 30,000 people in the United States (70,000 worldwide). Treating a complex disease like CF requires therapies that address problems in different parts of the body, especially the lungs and the digestive system. Because the type and severity of CF symptoms can differ widely from person to person, there is no typical treatment plan for people with the disease nor is there a cure.

These examples illustrate the need to develop new compositions to treat inflammation. Provided herein are solutions to these and other problems in the art.

### BRIEF SUMMARY OF THE INVENTION

Provided herein, inter alia, are thiocyanate salts and methods of using the same.

In one aspect, a salt comprising a cationic compound having the structure of Formula (I):

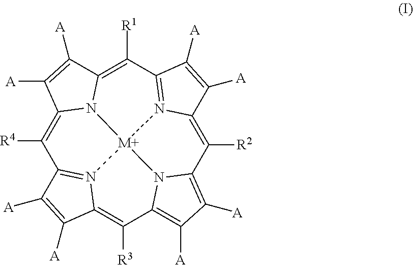

and an anionic compound having the structure of thiocyanate (⁻SCN). R¹, R², R³, and R⁴ are each independently

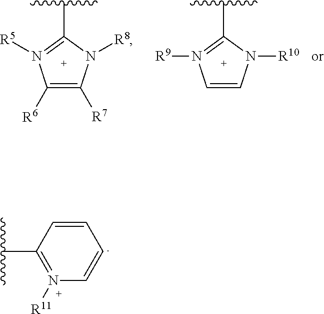

R⁵, R⁶, R⁷, R⁸, R⁹, and R¹⁰ are each independently hydrogen, halogen, —CN, —CF₃, —OH, —NH₂, —COOH, —COOR¹², —CH₂COOR¹², —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl. R¹¹ is —(CH₂)ₘCH₂OX¹ or —(CH₂CH₂O)ₙX¹; m is 0-6; n is 1-50. X¹ is substituted or unsubstituted C₁₋₁₂ alkyl. R¹² is an unsubstituted alkyl; M is a metal. Each A is independently hydrogen or an electron withdrawing group.

In another aspect, a pharmaceutical composition is provided including the thiocyanate salt of formula (I), including embodiments thereof, and a pharmaceutically acceptable excipient.

In another aspect, a method of treating inflammation in a subject in need thereof is provided. The method includes administering to the subject an effective amount of the thiocyanate salt of formula (I), including embodiments thereof.

In another aspect, a method of making hypothiocyanate is provided. The method includes contacting the thiocyanate salt as formula (I), including embodiments thereof, with hydrogen peroxide, thereby forming hypothiocyanate.

### BRIEF DESCRIPTION OF THE DRAWINGS

FIG. 1 is a bar graph showing the synergistic effects of AEOL 10150SCN salt in protecting human bronchial epithelial cells against hypochlorite-induced injury. Bars with different letters are significantly different, p<0.05. Bins (left to right): PBS (phosphate buffered saline); 250 uM HOCl; 250 uM HOCl+10 uM 10150-Cl; 250 uM HOCL+10 uM 10150-SCN; 250 uM HOCL+10 uM NaSCN.

FIG. 2 is a bar graph showing the SOD activities of 10150-Cl and 10150-SCN salts in the cytochrome c SOD assay. One unit of SOD is defined as the amount of compound that decreases superoxide cytochrome C reduction at abs 550nm by one half. Bins (left to right); Cmpd 10150-Cl; Cmpd 10150-SCN.

FIG. 3. is a line graph showing dose-dependent inhibition of rat brain lipid peroxidation of 10150-C1 and 10150-SCN salts as determined by measuring TBARS formation. The IC₅₀s are not statistically different, p=0.32. Legend: Cmpd 10150-Cl (circles); Cmpd 10150-SCN (boxes).

FIG. 4 is a line graph showing effects of 1 mM NaSCN on the consumption of H₂O₂ by 10150-Cl at a fixed concentration of 12.5 μM and various H₂O₂ concentrations. Legend: Cmpd 10150-Cl (circles); Cmpd 10150-Cl+SCN (1 mM) (triangles).

FIG. 5 is a bar graph showing the inhibition of oxygen formation by 10150-Cl in the presence of hydrogen peroxide and thiocyanate. Bins (left to right); 25 uM Cmpd 10150; 50 uM Cmpd 10150. Legend: control (open); SCN 1 mM (closed).

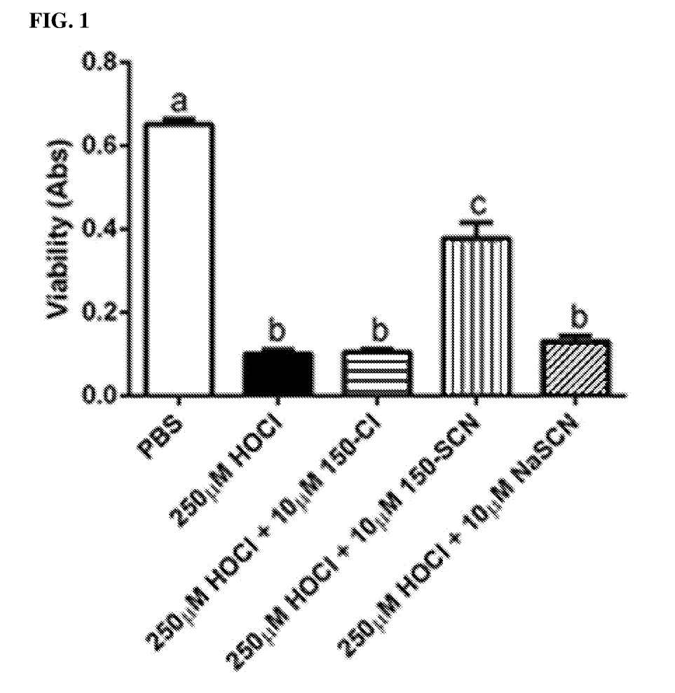

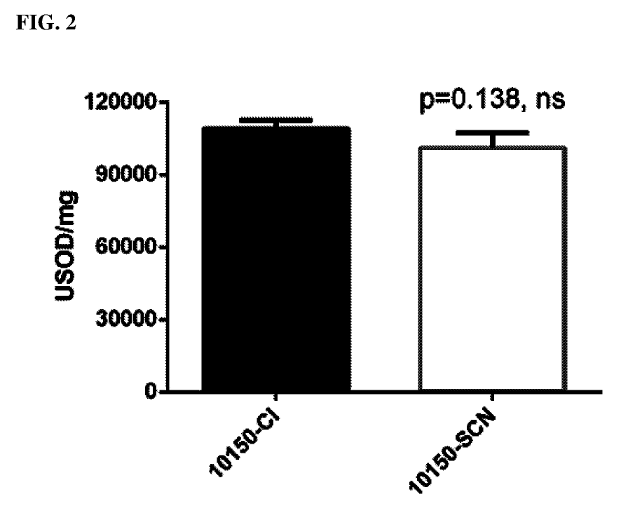

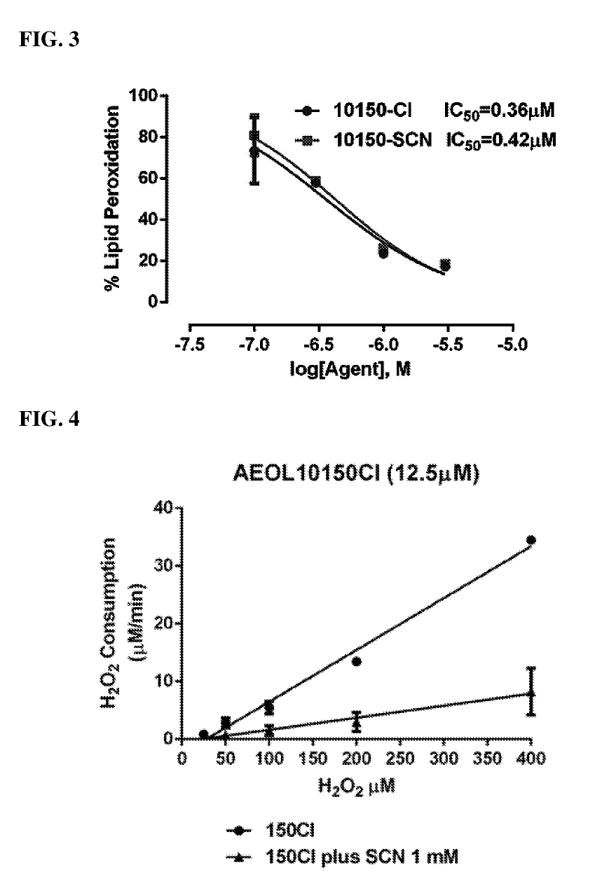

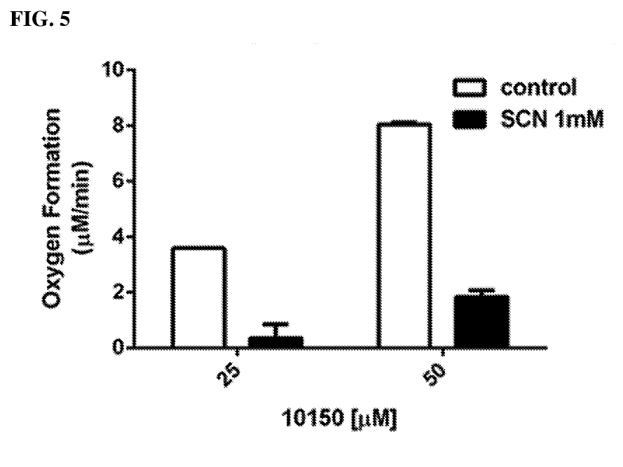

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). The term alkyl does not include cyclic alkyls. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g. selected from the group consisting of O, N, P, Si, and S, and wherein the nitrogen and sulfur atoms may optionally be oxidized, and the nitrogen heteroatom may optionally be quaternized). The heteroatom(s) O, N, P, S, B, As, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, non-aromatic cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C═(O)NR″NR″′R″″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R, R′, R′, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C═(O)NR″NR″′R″″, —CN, —NO₂, —R′, —N₃, —CH(Ph)₂, fluoro(C₁-C₄)alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R″′, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one or more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two or more substituents may optionally be joined to form aryl, heteroaryl, cycloalkyl, or heterocycloalkyl groups. Such so-called ring-forming substituents are typically, though not necessarily, found attached to a cyclic base structure. In embodiments, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R″′ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “ lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and/or each substituted or unsubstituted heterocycloalkyl is a substituted or unsubstituted 5 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₅-C₇ cycloalkylene, and/or each substituted or unsubstituted heterocycloalkylene is a substituted or unsubstituted 5 to 7 membered heterocycloalkylene.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to ionic compounds that result from the neutralization reaction of an acid and a base. They are composed of related numbers of cations (positively charged ions) and anions (negative ions) so that the product is electrically neutral (without a net charge). These component ions can be inorganic, such as chloride (Cl⁻), or organic, such as acetate (C₂H₃O₂⁻); and can be monatomic, such as fluoride (F⁻), or polyatomic, such as sulfate (SO₄²⁻). Illustrative examples of the present invention is a thiocyanate (also known as rhodanide) salt of a porphyrin, wherein the porphyrin is the cation and the thiocyanate [SCN]⁻ is the anion.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat diseases associated with inflammation. Certain methods described herein may treat diseases associated with inflammation (e.g., lung inflammation) by inhibiting estrogen receptor activity. Certain methods described herein may treat diseases associated with estrogen receptor activity by inhibiting ligand binding to estrogen receptor. Certain methods described herein may treat diseases associated with estrogen receptor activity by inducing the degradation of estrogen receptor. Certain methods described herein may treat diseases associated with inflammation by modulating enzyme activities of MPO and LPO. For example certain methods herein treat inflammation by decreasing a symptom of inflammation. Symptoms of inflammation would be known or may be determined by a person of ordinary skill in the art. The term “treating” and conjugations thereof, include prevention of an injury, pathology, condition, or disease.

An “effective amount” is an amount sufficient for a compound to accomplish a stated purpose relative to the absence of the compound (e.g. achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. inflammation) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with inflammation may be treated with an agent (e.g. compound as described herein) effective for decreasing inflammation.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g.

increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a harmful mediator/substance decreased in a disease. Activation may include, at least in part, partially or totally increasing stimulation, increasing or enabling activation, or activating, sensitizing, or up-regulating signal transduction or enzymatic activity or the amount of a harmful mediator/substance.

The term “modulator” refers to a composition that increases or decreases the level of a target molecule or the function of a target molecule. In embodiments, a modulator is an anti-inflammatory agent. In embodiments, a modulator is an inhibitor of MPO and/or LPO. In embodiments, a modulator is a SOD ligand.

“Anti-inflammatory agent” or anti-inflammatory refers to the property of a substance or treatment that reduces inflammation or swelling. Anti-inflammatory drugs make up about half of analgesics. There are nonsteroidal anti-inflammatory (NSAIDS) drugs (e.g., aspirin, ibuprofen, and naproxen) as well as steroids (e.g., prednisone, prednisolone, and dexamethasone) that can be used to treat inflammatory diseases or disorders.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with a compound, pharmaceutical composition, or method provided herein. In some embodiments, the disease is a disease having the symptom of cell proliferation. In some embodiments, the disease is an inflammation. In some further instances, “inflammation” refers to acute and chronic inflammation any tissue or organ within the human body. This inflammation may be the primary cause of the disease and/or disorder to be treated or may also by a result of the primary disease and/or disorder, which is non-inflammatory based.

As used herein, the term “lung inflammation” refers to inflammation of the lung tissue such as emphysema, asthma, ARDS including oxygen toxicity, pneumonia (especially

AIDS-related pneumonia), chronic obstructive pulmonary disease (COPD), emphysema, cystic fibrosis, bronchopulmonary dysplasia, chronic sinusitis, arthritis and autoimmune diseases (such as lupus or rheumatoid arthritis) and pulmonary fibrosis (e.g. Idiopathic Pulmonary Fibrosis (IPF), Idiopathic Interstitial Pneumonia (IIP), Interstitial Lung Disease (ILD). Sarcoidosis, Lymph-angioleimyomatosis (LAM), Wegener's Granulomatosis).

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated

Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-inflammatory agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### II. Compounds

Provided herein, inter alia, are compositions of a salt including a cationic compound having the structure of Formula (I):

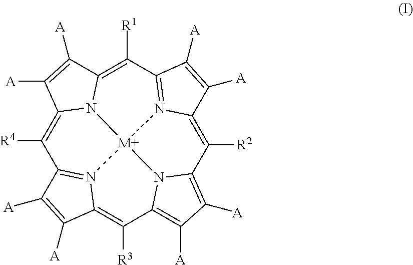

and an anionic compound having the structure of ⁻SCN. Thus, the salt may include at least one thiocyanate. In embodiments, the salt includes one thiocyanate anionic compounds. In embodiments, the salt includes two thiocyanate anionic compounds. In embodiments, the salt includes three thiocyanate anionic compounds. In embodiments, the salt includes four thiocyanate anionic compounds. In embodiments, the salt includes four thiocyanate anionic compounds. In addition to a thiocyanate anionic compounds, in embodiments, the salt includes an additional anionic compound that is not thiocyanate, such as chlorine, fluoride, sulfide, a sulfate, a carbonate, and/or a phosphate. In embodiments, additional anionic compound that is not thiocyanate is chlorine.

R¹, R², R³, and R⁴ are each independently

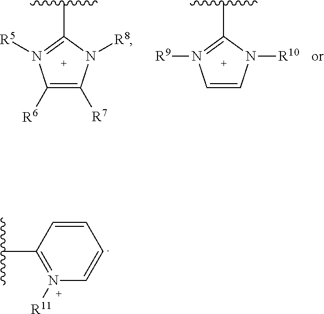

R⁵, R⁶, R⁷, R⁸, R⁹, and R¹⁰ are each independently hydrogen, halogen, —CN, —CF₃, —OH, —NH₂, —COOH, —COOR¹², —CH₂COOR¹², —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl.

R¹¹ is —(CH₂)ₘCH₂OX¹ or —(CH₂CH₂O)ₙX¹. The symbol m is an integer from 0 to 6. The symbol n is an integer from 1 to 50. The symbol X¹ is substituted or unsubstituted C₁₋₁₂ alkyl. The symbol R¹² is an unsubstituted alkyl. The symbol M is a metal. Each symbol A is independently hydrogen or an electron withdrawing group.

In embodiments of formula (I), the metal is manganese, iron, cobalt, copper, nickel or zinc.

In embodiments of formula (I) the metal is manganese. In embodiments, the manganese is a manganese (III). In embodiments, the manganese is a manganese (II).

In embodiments, R¹, R², R³, and R⁴ are each

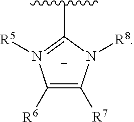

R⁵, R⁶, R⁷, and R⁸ may each independently be hydrogen, halogen, —CN, —CF₃, —OH, —NH₂, —COOH, —COOR¹², —CH₂COOR¹², —CH₂COOH, R¹³-unsubstituted or substituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl. R¹³ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl. R¹⁴ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) the metal is manganese. R¹⁴ may be C₁-C₅ alkyl.

In embodiments of formula (I) the metal is manganese. R¹, R², R³, and R⁴ may each be

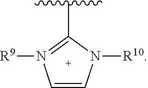

R⁹ and R¹⁰ may each independently be hydrogen, halogen, —CN, —CF₃, —OH, —NH₂, —COOH, —COOR¹², —CH₂COOR¹², —CH₂COOH, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl. R¹³ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl. R¹⁴ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) R¹⁴ is C₁-C₅ alkyl.

In embodiments of formula (I) the metal is manganese. R⁹ and R¹⁰ may each be unsubstituted ethyl.

In embodiments of formula (I) the metal is manganese. A may be hydrogen.

In embodiments of formula (I) the compound has the structure

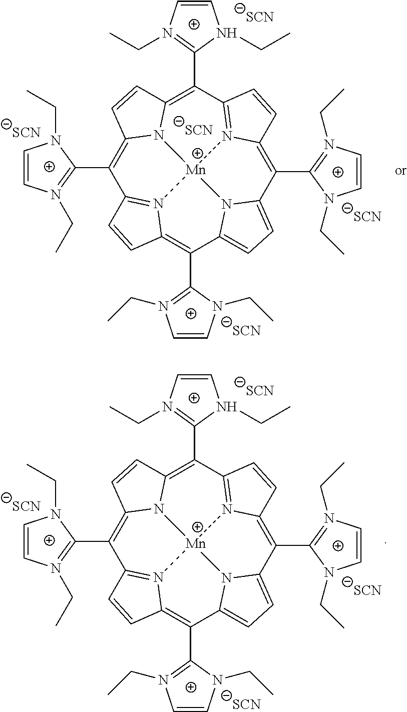

In embodiments of formula (I) the metal is manganese. R¹, R², R³, and R⁴ may each be

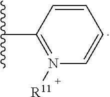

In embodiments of formula (I) the metal is manganese. R¹, R², R³, and R⁴ may each be

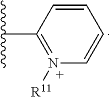

R¹¹ is —(CH₂)ₘCH₂OX¹ and m is 1-6.

In embodiments of formula (I) the metal is manganese. R¹, R², R³, and R⁴ may each be

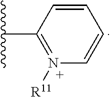

R¹¹ is —(CH₂CH₂O)ₙX¹ and n is 3-50.

In embodiments of formula (I) the metal is manganese. R¹, R², R³, and R⁴ may each be

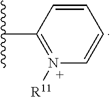

X¹ may be R¹³-substituted or unsubstituted alkyl. R¹³ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl. R¹⁴ may be halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

In embodiments of formula (I) the metal is manganese. R¹⁴ may be C₁-C₅ alkyl.

In embodiments of formula (I) the metal is manganese. X¹ may be C₁₋₅ alkyl.

In embodiments of formula (I) the metal is manganese. A may be hydrogen.

### III. Methods of Treatment

The salts described above, can be used in the treatment of inflammatory diseases and/or disorders by administering an effective amount of a salt as described to a subject in need thereof. The salts as described above exhibit anti-inflammatory action and anti-microbial action to boost the host's defense system while reducing tissue inflammation. The anti-inflammatory action is due to the effects of the manganese metalloporphyrin (e.g. structures of formula (I)), while the anti-microbial action is due to the thiocyanate (SCN), which competes with the cellular processes in inflammation to generate hypothiocyanate (HOSCN), an anti-inflammatory mediator.

The antioxidant and anti-inflammatory actions of the salts of the invention are due to the manganese metalloporphyrin, which hampers the innate immunity by scavenging oxidants used by the innate immune system to kill pathogens. However, inflammatory mediators are released from the pathogens before their demise, stimulating inflammatory pathways. Thiocyanate can interfere with these inflammatory pathways by generating hypothiocyanate (HOSCN). The formation of hypothiocyanate (HOSCN) protects the host against other immune mediated halous acid (HOCl) induced injury as well as decrease the host's inflammation due to selective metabolism of HOSCN by the host but not by the pathogen. Hypothiocyanate has also been recently found to be selectively detoxified by mammalian thioredoxin reductase but not bacterial thioredoxin reductase providing a mechanism by which formation of hypothiocyanate retains host defense while limiting host damage and inflammation through selective host metabolism of HOSCN (Chandler et al. Journal of Biological Chemistry 288:18421-18428, 2013).

The salts of the present invention demonstrate anti-oxidant properties by exhibiting superoxide dismutation (SOD) activity. Superoxide dismutation (SOD) activity of the salts of the invention was assessed using the xanthine oxidase/cytochrome c assay which measures the ability of a compound or enzyme to compete with cytochrome c for reaction with superoxide spectrophotometrically at 550 nm.

The ability of the salts of the invention to inhibit lipid peroxidation was assessed as described by Ohkawa et al. (Anal. Biochem. 95:351 (1979)) and Yue et al. (J. Pharmacol. Exp. Ther. 263:92 (1992)). Iron and ascorbate can be used to initiate lipid peroxidation in tissue homogenates and the formation of thiobarbituric acid reactive species (TBARS) measured. Lipid peroxidation causes cell damage by radicals, which destroys lipids in cell membranes resulting in oxidative and inflammatory injury.

The salts of the present invention modulate the activity of the enzyme catalase, which catalyzes the decomposition of hydrogen peroxide to water and oxygen. Hydrogen peroxide is a substrate for the enzyme myeloperoxidase (MPO), which produces hypochlorous acid (HOCl), a highly damaging oxidant with anti-bacterial properties.

The salts of the present invention modulates the catalase activity, wherein the thiocyanate competes as a substrate of the catalyze enzyme to generate HOSCN and not HOC1 in the conversion of hydrogen peroxide.

The salts of the present invention also modulate lactoperoxidase (LPO) activity, wherein the enzyme catalyzes hydrogen peroxide (H₂O₂) oxidation of several acceptor molecules including thiocyanate to generate hypothiocyante.

When inflammation occurs, MPO and LPO are released by neutrophils, which migrate towards the site of inflammation. Neutrophils are one of the first-responders of inflammatory cells and migrate towards the site of inflammation to release inflammatory mediators such as cytokines, which in turn amplify inflammatory reactions of other cell types.

The salts of the present invention are used in the treatment of diseases or disorders associated with elevated levels and/or activities of these enzymes describes above. The salts are further preferred for use in the treatment of diseases or disorders mediated by oxidative stress such as inflammatory diseases, particularly inflammation of the lungs.

The salts of the present invention can be used in to treat lung inflammation, which are caused by a virus or bacteria. For example, pneumonia and asthma may be caused by a bacteria (e.g., Streptococcus pneumoniae, Haemophilus influenzae, Chlamydophila pneumoniae, Mycoplasma pneumoniae, Staphylococcus aureus; Moraxella catarrhalis; Legionella pneumophila and Gram-negative bacilli) or virus (e.g., rhinoviruses, coronaviruses, influenza virus, respiratory syncytial virus (RSV), adenovirus, and parainfluenza). The salts of the present invention can also be used in to treat lung inflammation, wherein the virus or bacteria is resistant to antibiotics and antivirals.

As indicated above, inflammations, particularly inflammations of the lung, are amenable to treatment using the present salts such as inflammatory based disorders of emphysema, asthma, ARDS including oxygen toxicity, pneumonia (especially AIDS-related pneumonia), chronic obstructive pulmonary disease (COPD), emphysema, cystic fibrosis, bronchopulmonary dysplasia, chronic sinusitis, arthritis and autoimmune diseases (such as lupus or rheumatoid arthritis) and pulmonary fibrosis (e.g. Idiopathic Pulmonary Fibrosis (IPF), Idiopathic Interstitial Pneumonia (IIP), Interstitial Lung Disease (ILD). Sarcoidosis, Lymph-angioleimyomatosis (LAM), Wegener's Granulomatosis).

In another aspect, a method of treating inflammation in a subject in need thereof is provided. The method includes administering to the subject an effective amount of the thiocyanate salt of formula (I), including embodiments thereof. In embodiments, the inflammation is lung inflammation (inflammation in the lungs). In embodiments, the inflammation is, or is the result of, an inflammatory based disorder of cystic fibrosis, asthma, chronic obstructive pulmonary disease (COPD), pneumonia, emphysema, respiratory distress syndrome (ARDS), or bronchopulmonary dysplasia. In embodiments, the bacteria or virus is resistant to antibiotics and antivirals, respectively. In embodiments, the inflammation activates neutrophils to release enzymes MPO and LPO. In embodiments, the methods provided the thiocyanate salt of formula (I), including embodiments thereof, wherein said salt inhibits LPO activity, generates an antioxidant, and decreases inflammation.

In another aspect, a method of making hypothiocyanate is provided. The method includes contacting the thiocyanate salt as formula (I), including embodiments thereof, with hydrogen peroxide, thereby forming hypothiocyanate.

In another aspect, a method of treating an injury (e.g. inflammation) associated with an organ in a subject in need thereof is provided. The organ may be skin, lungs, nose, esophagus, trachea, or bronchi. In embodiments, the organ is a lung. The agent causing the injury may be a nitrogen mustard agent, including mechlorethamine hydrochloride, chlorambucil, busulfan, cyclophosphamide, and the sulfur mustards including chlorine gas, phosgene, and 2-chloroethyl ethyl sulfide. In embodiments, the agent may be chosen from a sulfur mustard gas, chlorine gas, phosgene, and 2-chloroethyl ethyl sulfide. In embodiments, the agent is a sulfur mustard. In embodiments, the agent is a chlorine gas. In embodiments, when a subject has been exposed to chlorine gas, administration of the salts of the invention can decrease damage to the tissue, specifically lung tissue, by preventing the formation of harmful hypochlorite (HOCl) molecules upon contact with water present in the tissue and rather promote formation of hypothiocyanous acid (HOSCN), an anti-inflammatory species, thereby preventing damage to the tissue.

### IV. Pharmaceutical Compositions

The salts described above, can be formulated into pharmaceutical compositions suitable for use in the present methods. Such compositions include the active agent (thiocyanate salts of metalloporphyrin compounds) together with a pharmaceutically acceptable carrier, excipient or diluent.

In another aspect, a pharmaceutical composition includes the thiocyanate salt of formula (I) and a pharmaceutically acceptable excipient.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting inflammation. Determination of a therapeutically effective amount of a salt of the invention is well within the capabilities of those skilled in the art, especially in light of the detailed disclosure herein.

For preparing pharmaceutical compositions from the salt of the present invention, pharmaceutically acceptable carriers can be either solid or liquid. Solid form preparations include powders, tablets, pills, capsules, cachets, suppositories, and dispersible granules. A solid carrier can be one or more substances, that may also act as diluents, flavoring agents, binders, preservatives, tablet disintegrating agents, or an encapsulating material.

In powders, the carrier is a finely divided solid in a mixture with the finely divided active component (e.g. a compound provided herein). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the salts of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The salts of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active salt (e.g. compounds described herein, including embodiments, and examples) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The salts of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce inflammation).

The salts of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The salts of the present invention can also be administered by injection, that is, intravenously, intramuscularly, intracutaneously, subcutaneously, intraduodenally, or intraperitoneally. Also, the salts described herein can be administered by inhalation, for example, intranasally. Additionally, the salts of the present invention can be administered transdermally. It is also envisioned that multiple routes of administration (e.g., intramuscular, oral, transdermal) can be used to administer the salts of the invention. Accordingly, the present invention also provides pharmaceutical compositions comprising a pharmaceutically acceptable excipient and one or more salts of the invention.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component.

The composition can, if desired, also contain other compatible therapeutic agents. Some compounds may have limited solubility in water and therefore may require a surfactant or other appropriate co-solvent in the composition. Such co-solvents include: Polysorbate 20, 60 and 80; PLURONIC® F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., emphysema, asthma, ARDS including oxygen toxicity, pneumonia, chronic obstructive pulmonary disease (COPD), emphysema, cystic fibrosis, bronchopulmonary dysplasia, chronic sinusitis, pulmonary fibrosis), kind of concurrent treatment, complications from the disease being treated or other health-related problems. The disease may be a primary inflammatory disease and/or disorder. The disease may be a caused by a primary non-inflammatory disorder resulting in an inflammatory disease and/or disorder. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In embodiments, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration and dosage can be chosen by the individual physician in view of the patient's condition and the particular method in which the compound is used

### IV. EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1

[5,10,15,20 tetrakis(1,3-diethylimidazolium-2-yl)porphyrinato] manganese (III) pentathiocyanate (10150-SCN) was found to have synergistic effects when compared to either the [5,10,15,20 tetrakis(1,3-diethylmidazolium-2-yl)porphyrinato] manganese (III) pentachloride (10150-Cl) or sodium thiocyanate in (NaSCN) providing protection against hypochlorite-mediated epithelial cell injury.

Human bronchial epithelial cells (HBE) were exposed to 250 μM of hypochlorite (HOCl) for 30 minutes in the presence or absence of 10 μM of 10150-Cl, 10150-SCN, or NaSCN and cell injury was assessed 24 hours post HOCl exposure using the MTT viability assay.

### Example 2

Both the chloride and thiocyanate salts of 10150 have similar superoxide dismutase activity. Superoxide dismutation (SOD) activity was assessed using the xanthine oxidase/cytochrome c assay which measures the ability of a compound or enzyme to compete with cytochrome c for reaction with superoxide spectrophotometrically at 550 nm.

### Example 3

Both the chloride and thiocyanate salts of 10150 have similar lipid peroxide inhibition. Lipid peroxidation of rat brain homogenates was initiated with iron/ascorbate in the presence or absence of various concentrations of 10150-Cl or 10150-SCN. Lipid peroxidation was quantified using the TBARS assay with malondialdehyde standards. The data was normalized and curve fitted to determine the inhibitory concentrations that decreased lipid peroxidation by one-half (IC₅₀). Both compounds exhibited similar IC₅₀s in the rat brain homogenates.

### Example 4

10150-Cl has haloperoxidase activity using thiocyanate as a substrate generating HOSCN. 10150-Cl is known to possess catalase activity which is the dismutation of hydrogen peroxide into oxygen and water (see Eq. 1).

2H₂O₂→O₂+2H₂O Eq. 1

When comparing the 10150-Cl and SCN salts it was noticed that the 10150-SCN had lower catalase activity than 10150-Cl salt. To test whether SCN was acting as a competing substrate in the catalase activity one examined the change in 10150-Cl catalase activity by following the disappearance of H₂O₂ over time with a H₂O₂ selective electrode in the presence or absence of 1 mM NaSCN. It was found that SCN decreased the rate of H₂O₂ disappearance.

Experimental Conditions: Effects of 1 mM NaSCN on the consumption of H₂O₂ by 10150-Cl was investigated at a fixed concentration of 12.5 μM and various H₂O₂ concentrations. H₂O₂ consumption was followed over time with a hydrogen peroxide selective electrode (HPO) using a free radical analyzer (WPI). Changes in pA were converted to H₂O₂ using a 5 point H₂O₂ standard curve. The data was fitted as a linear curve using Prizm software. The rate constant for 10150-Cl was 0.09±0.004 min⁻¹ and in the presence of 1 mM NaSCN was decreased to 0.02±0.004 min⁻¹.

### Example 5

It was examined whether SCN was being utilized as a substrate in a peroxidase reaction by looking at the inhibition of oxygen formation. If 10150-Cl was utilizing SCN along with H₂O₂, then no oxygen would be generated (see Eq 2).

H₂O₂+2SCN→2HOSCN Eq 2

To examine the rate of oxygen formation in the presence of 10150-Cl, H₂O₂ in the presence or absence of NaSCN, one utilized an oxygen selective electrode with a free radical analyzer (WPI). Changes in pA were converted to O₂ using the difference of the O₂ concentration in a saturated solution minus that of the solution made anaerobic by the addition of dithionite.

Experimental Conditions: Conversion of catalase activity to haloperoxidase activity of 10150-Cl with the addition of thiocyanate (SCN). Oxygen formation was followed with an oxygen selective electrode in a reaction mixture containing 10150 at either 25 or 50 μM and 1 mM H₂O₂ in the presence or absence of 1 mM SCN. The addition of SCN dramatically inhibited the formation of oxygen supporting the conversion of hydrogen peroxide dismutation to hypothiocyanate formation.

Hypothiocyanate has been recently found to be selectively detoxified by mammalian thioredoxin reductase but not bacterial thioredoxin reductase providing a mechanism by which formation of hypothiocyanate retains host defense while limiting host damage and inflammation through selective host metabolism of HOSCN (Chandler et al. Journal of Biological Chemistry 288:18421-18428, 2013).

### V. EMBODIMENTS

Embodiments contemplated herein include the following.

### Embodiment 1

A salt comprising a cationic compound having the structure of Formula (I):

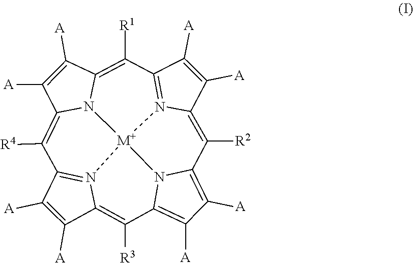

and an anionic compound having the structure of ⁻SCN; wherein R¹, R², R³, and R⁴ are each independently

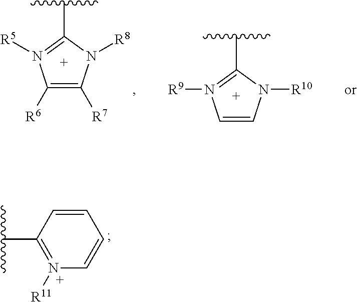

R⁵, R⁶, R⁷, R⁸, R⁹, and R¹⁰ are each independently hydrogen, halogen, —CN, —CF₃, —OH, —NH₂, —COOH, —COOR¹², —CH₂COOR¹², —CH₂COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl; R¹¹ is —(CH₂)ₘCH₂OX¹ or —(CH₂CH₂O)ₙX¹; m is 0-6; n is 1-50; X¹ is substituted or unsubstituted C₁₋₁₂ alkyl; R¹² is an unsubstituted alkyl; M is a metal; and each A is, independently hydrogen or an electron withdrawing group.

### Embodiment 2

The salt of embodiment 1, wherein the metal is selected from the group consisting of manganese, iron, cobalt, copper, nickel, and zinc.

### Embodiment 3

The salt of embodiment 2, wherein the metal is manganese.

### Embodiment 4

The salt of embodiment 3, wherein R¹, R², R³, and R⁴ are each

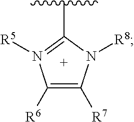

R⁵, R⁶, R⁷, and R⁸ are each independently hydrogen, halogen, —CN, —CF₃, —OH, —NH₂, —COOH, —COOR¹², —CH₂COOR¹², —CH₂COOH, R¹³-unsubstituted or substituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl; R¹³ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl; and R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

### Embodiment 5

The salt of embodiment 4, wherein R¹⁴ is C₁-C₅ alkyl.

### Embodiment 6

The salt of embodiment 3, wherein R¹, R², R³, and R⁴ are each

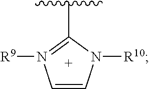

R⁹ and R¹⁰ are each independently hydrogen, halogen, —CN, —CF₃, —OH, —NH₂, —COOH, —COOR¹², —CH₂COOR¹², —CH₂COOH, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl; R¹³ is halogen, —NH₂, —CF₃, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl; and R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

### Embodiment 7

The salt of embodiment 6, wherein R¹⁴ is C₁-C₅ alkyl.

### Embodiment 8

The salt of embodiment 6, wherein R⁹ and R¹⁰ are each unsubstituted ethyl.

### Embodiment 9

The salt of embodiment 8, wherein A is hydrogen.

### Embodiment 10

The salt of embodiment 1 having the structure

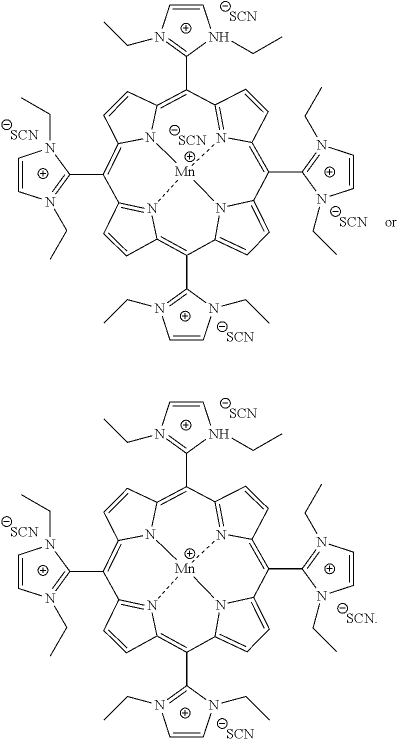

### Embodiment 11

The salt of embodiment 3, wherein R¹, R², R³, and R⁴ are each

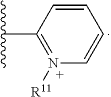

### Embodiment 12

The salt of embodiment 11, wherein R¹¹ is —(CH₂)ₘCH₂OX¹; and m is 1-6.

### Embodiment 13

The salt of embodiment 11, wherein R^{H} is —(CH₂CH₂O)ₙX¹; and n is 3-50.

### Embodiment 14

The salt as in embodiment 12 or 13, wherein X¹ is R¹³-substituted or unsubstituted alkyl; R¹³ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, substituted or unsubstituted heterocycloalkyl, R¹⁴-substituted or unsubstituted aryl, or an R¹⁴-substituted or unsubstituted heteroaryl; and R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

### Embodiment 15

The salt of embodiment 14, wherein R¹⁴ is C₁-C₅ alkyl.

### Embodiment 16

The salt as in embodiment 12 or 13, wherein X¹ is C₁₋₅ alkyl.

### Embodiment 17

The salt of embodiment 16, wherein A is hydrogen.

### Embodiment 18

A pharmaceutical composition comprising a salt as in one of embodiments 1-17 and a pharmaceutically acceptable excipient.

### Embodiment 19

A method of treating inflammation in a subject in need thereof, comprising administering to said subject an effective amount of a salt as in one of embodiments 1-17.

### Embodiment 20

The method of embodiment 19, wherein said inflammation is an inflammation of the lungs.

### Embodiment 21

The method of embodiment 19, wherein said inflammation is an inflammatory based disorder of cystic fibrosis, asthma, chronic obstructive pulmonary disease (COPD), pneumonia, emphysema, respiratory distress syndrome (ARDS), or bronchopulmonary dysplasia.

### Embodiment 22

The method of embodiment 19, wherein said inflammation is caused by a virus or bacteria.

### Embodiment 23

The method of embodiment 22, wherein said virus or bacteria is resistant to antibiotics and antivirals.

### Embodiment 24

The method of embodiment 19, wherein said inflammation activates neutrophils to release enzymes MPO and LPO.

### Embodiment 25

The method of embodiment 19, wherein a salt of embodiment 1 inhibits LPO activity, generating an antioxidant, and decreasing inflammation.

### Embodiment 26

A method of making hypothiocyanate, said method comprising contacting the salt as in one of embodiments 1-17 with hydrogen peroxide, thereby forming hypothiocyanate.

1. A salt comprising a cationic compound having the structure of Formula (I): 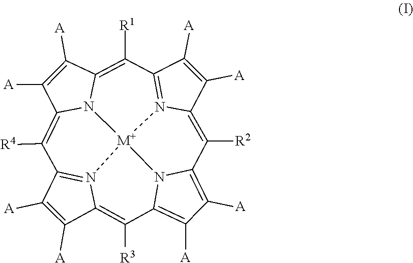
and an anionic compound having the structure of ⁻SCN;
wherein
R¹, R², R³, and R⁴ are each independently 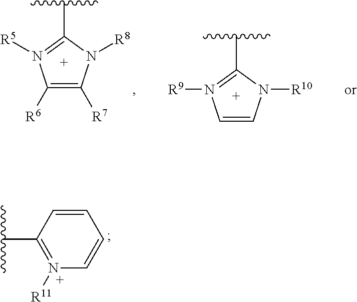
R⁵, R⁶, R⁷, R⁸, R⁹, and R¹⁰ are each independently hydrogen, halogen, —CN, —CF₃, —OH, —NH₂, —COOH, —COOR¹², —CH₂COOR¹², —CH2COOH, an unsubstituted or substituted alkyl, unsubstituted or substituted heteroalkyl, unsubstituted or substituted cycloalkyl, unsubstituted or substituted heterocycloalkyl, unsubstituted or substituted aryl, or an unsubstituted or substituted heteroaryl;
R¹¹ is —(CH₂)ₘCH₂OX¹ or —(CH₂CH₂O)ₙX¹;
m is 0-6;
n is 1-50;
X¹ is substituted or unsubstituted C₁₋₁₂ alkyl;
R¹² is an unsubstituted alkyl;
M is a metal; and
each A is, independently hydrogen or an electron withdrawing group.

2. The salt of claim 1, wherein the metal is selected from the group consisting of manganese, iron, cobalt, copper, nickel, and zinc.

3. (canceled)

4. The salt of claim 2, wherein R¹, R², R³, and R⁴ are each 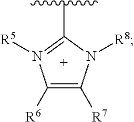
R⁵, R⁶, R⁷, and R⁸ are each independently hydrogen, halogen, —CN, —CF₃, —OH, —NH₂, —COOH, —COOR¹², —CH₂COOR¹², —CH₂COOH, R¹³-unsubstituted or substituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl;
R¹³ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl; and
R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

5. The salt of claim 4, wherein R¹⁴ is C₁-C₅ alkyl.

6. The salt of claim 3, wherein R¹, R², R³, and R⁴ are each 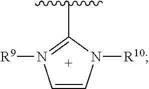
R⁹ and R¹⁰ are each independently hydrogen, halogen, —CN, —CF₃, —OH, —NH₂, —COOH, —COOR¹², —CH₂COOR¹², —CH₂COOH, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or an R¹³-substituted or unsubstituted heteroaryl;
R¹³ is halogen, —NH₂, —CF₃, —CHF₂, —CH₂F, —CN, —SO₂NH₂, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl; and
R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

7. The salt of claim 6, wherein R¹⁴ is C₁-C₅ alkyl and R⁹ and R¹⁰ are each unsubstituted ethyl.

8. (canceled)

9. The salt of claim 8, wherein A is hydrogen.

10. The salt of claim 1 having the structure

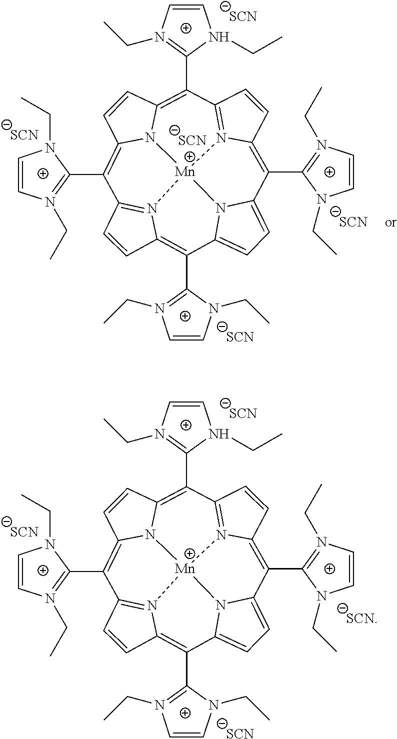

11. The salt of claim 3, wherein R¹, R², R³, and R⁴ are each 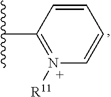
wherein R¹¹ is —(CH₂)ₘCH₂OX¹; and m is 1-6, or R¹¹ is —(CH₂CH₂O)ₙX¹; and n is 3-50.

12. (canceled)

13. (canceled)

14. The salt as in claim 11, wherein X¹ is R¹³-substituted or unsubstituted alkyl;
R¹³ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC (O)H, —NHC(O)OH, —NHOH, —OCF₃, oxo, —N₃, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or an R¹⁴-substituted or unsubstituted heteroaryl; and
R¹⁴ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

15. The salt of claim 14, wherein R¹⁴ is C₁-C₁₅ alkyl.

16. The salt as in claim 11, wherein X¹ is C₁₋₅ alkyl.

17. The salt of claim 16, wherein A is hydrogen.

18. A pharmaceutical composition comprising a salt of claim 1 and a pharmaceutically acceptable excipient.

19. A method of treating inflammation in a subject in need thereof, comprising administering to said subject an effective amount of a salt of claim 1.

20. The method of claim 19, wherein said inflammation is an inflammation of the lungs or an inflammatory based disorder of cystic fibrosis, asthma, chronic obstructive pulmonary disease (COPD), pneumonia, emphysema, respiratory distress syndrome (ARDS), or bronchopulmonary dysplasia.

21. (canceled)

22. The method of claim 19, wherein said inflammation is caused by a virus or bacteria resistant to antibiotics and antivirals.

23. (canceled)

24. The method of claim 19, wherein said inflammation activates neutrophils to release enzymes MPO and LPO.

25. The method of claim 19, wherein a salt of claim 1 inhibits LPO activity, generating an antioxidant, and decreasing inflammation.

26. A method of making hypothiocyanate, said method comprising contacting a salt of claim 1 with hydrogen peroxide, thereby forming hypothiocyanate.
